(19) United States
(12) Patent Application Publication
(10) Pub. No.: US 2017/0049805 A1
(43) Pub. Date: Feb. 23, 2017

(54) PROCESS FOR SUSTAINED NEUROMODULATION OF THE NERVOUS SYSTEM

(71) Applicant: Transfer Devices, Inc., Fremont, CA (US)

(72) Inventor: Charles Schaper, Union City, CA (US)

(73) Assignee: Transfer Devices, Inc., Fremont, CA (US)

(21) Appl. No.: 15/190,423

(22) Filed: Jun. 23, 2016

Related U.S. Application Data
(60) Provisional application No. 62/151,791, filed on Apr. 23, 2015.

(51) Int. Cl. A61K 31/765; A61K 9/00; A61K 9/70

(52) U.S. Cl. CPC A61K 31/765; A61K 9/70; A61K 9/0014

(57) ABSTRACT

A water soluble polymer thin film of polyvinyl alcohol is manufactured and placed in contact with the skin, or through a buffering layer of cream, and then lightly sprayed on the exposed side with water to achieve conformal contact with the skin microstructures by transition from a solid state to a partial liquid/solid or gel state, and then upon drying back to a solid state. As the polymer thin film dries it contracts over a prolonged period, thereby producing a tightening effect of sustained tensile and compressive forces propagating through the skin surface layers with concurrent modulation of sensory nerve endings of the peripheral nervous system, with certain therapeutic effects depending upon the applied area, including reducing pain associated with arthritis or soreness of joint areas, inducing a relaxed and attentive cognitive state, inducing inflammation control or relief of cellular hydrostatic pressure, and reducing gastric reflex.

### CROSS-REFERENCE TO RELATED APPLICATION

The present application claims priority under 35 U.S.C. 119(e) from prior U.S. provisional application 62/151,791, filed Apr. 23, 2015. A petition is being filed to restore the benefit of the U.S. provisional application, pursuant to Public Law 112-211, §201(c) [Patent Law Treaties Implementation Act of 2012], 35 U.S.C. 119(e)(1), and 37CFR §1.78(b).

### TECHNICAL FIELD

This invention relates to improving physical and mental health through noninvasive processes involving the neuro-stimulation of cutaneous afferents of the nervous system.

### BACKGROUND ART

The nervous system, through a network involving signal receptors and transmitters, enables functional actions throughout the body along different pathways, as indicated in FIG. 1, including in an automatic capacity to many muscle and organs. Through disease, injury, as well as normative decline over time, the disruption of the nervous system structure itself, or to the structures that are under its control, can result in a significant reduction in the quality of life and the inability to produce maximal inherent function. In such cases, from neural plasticity and other mechanisms of self-healing, there is an adaptive capability to attenuate the effects of the reduced capacity by reorganizing or masking the impact. But without external intervention, such adaptive processes have limited effectiveness in extent and specificity, and over the course of a lifetime vary in their adaptive capability, becoming less proactive in the later stages of life.

It is known that during a body's growth period, cellular division requires continual innervation and connectivity of nerve tissue by relays throughout the body so as to communicate the present state of organic growth in order to coordinate activities, including those of both conscious and autonomic nature. In particular, the sensory system of the peripheral nervous system (PNS) provides stimulation beginning at nerve endings and has direct access to organs, in addition to triggering central nervous system (CNS) activity. But over time, any decrease or cessation of cellular division is hypothesized to cause a loss of stimulation of the corresponding nerve connections, because it is no longer required, and therefore can go dormant. The reduction or loss of interconnectivity between the sensory system and the organs may therefore lead to misprocessing in an otherwise healthy organ. For example, the sensory system may send a signal, or no signal, inadvertently to an organ, which also receives signals directly from the CNS. This lack of sensory feedback can cause poor functioning of the organs. Consequently, it is necessary to develop a method to maintain the communication channels of the PNS to the target organs and to the CNS. Any successful methodology should as much as possible mimic the process of nervous system interactions that naturally occurs during cellular division, to keep them from going dormant or to reactivate already dormant channels.

To improve the control of the nervous system, external access has been sought through invasive means, which includes surgical repair of the central nervous system, as well as non-invasive means, which includes acoustically, optically and electrically induced signals along the pathways of the Peripheral Nervous System (PNS). Such non-invasive methods of neuro-stimulation of the PNS seek to conveniently improve its capability, sometimes with specificity by direct control over the organ, as in a pacemaker, or over broad regions for example by control of inflammation through nerve stimulation. Such methods tend to provide excitation through an instantaneous signal, such as an electrical burst, for direct control and response.

A polymer film manufactured from polyvinyl alcohol (PVA) is available. Because this film contains both hydrophilic capability through alternating hydroxyl side-chains and hydrophobic associations because of a linear carbon backbone, as well as having physical attributes of strength and mobility, it is thereby capable of conformal contact with the microstructures of the skin surface. Previously, PVA films have been applied to skin for short time periods as part of certain cosmetic applications, such as to smooth the appearance of age-related wrinkles by the delivery of cosmetic agents.

### SUMMARY DISCLOSURE

A thin film polymer that is water soluble, conformal, and constricts when dried achieves various health-related effects due to (1) mechanical interaction to noninvasively produce tensile and compressive forces propagating through the skin layers created by the surface area rearrangement of the drying polymer film over a sustained period, and consequently (2) interaction with the sensory division of the peripheral nervous system. In particular, a thin film of polyvinyl alcohol (PVA) of sufficient size, for example a four-inch square, is placed on the surface of the target tissue, for example skin, and then a spray of water is applied to the film, which causes the film to come into conformal contact with the skin, forming tight interaction at the cellular layer. Upon drying, the PVA film produces tensile and compressive forces that cause the tissue to have the association of constriction, inducing a neuromodulation effect, in which electrochemical signals are sent from the affected area to produce a visceral sensation and stimulation. The PVA films can be placed throughout areas of body, and left for a period of time, between 15-120 minutes, to achieve the necessary neuromodulation, and then simply washed or peeled off the skin. Because of the thin film nature of the PVA film, and its ability to be safely handled easily and manufactured at a low cost, as well as biocompatibility, the noninvasive approach furthers its feasibility. Involving sustained spatial stimulation of the peripheral and autonomic nervous system, accessed over large areas of the sensory division, the technique can produce advantageous effects by the amplification of the multitude of interconnections within the central nervous system, autonomic nervous system, glands and tissues.

A wide variety of therapies and treatments can be developed using this basic concept, including:
The treatment of skin disorders such as acne, allowing deeper penetration of cleansing due to the effect of the PVA film to deliver the materials to the pore sites, and the stimulation of the skin by thin film constricting effects.
In addition, the treatment of open cuts by a constricting polymer film, in which the large thin film is placed over the wound, and during the constrictive drying phase induces an analgesic effect to reduce the pain and promote healing.
In areas where blood flow is low, such as in the tendons and joints, to promote healing the constrictive nature of the thin film polymer will enable a response that requires the increase of blood flow to enable mechanoreceptors to send signals.
The addition to topical medications to the thin film polymer can result in the transfer of material to the surface of the skin.
Because of its location with access to the facial nerve system, depending upon the level of tensile strength of the thin polymer film, it is possible to address and treat issues associated with facial nerves.
Treatment of cognitive abilities through control of the peripheral nervous system.
Treatment of ability to self-heal damaged nerves by stimulation over an extended period of time.
Treatment to stimulate the interaction and control of nerve cells to muscle cells, despite losses in the sensitivity of the muscle spindle with age, in addition to losses in muscle fibers, and the possible polyneural innervation that require adaptation to the new nerve-muscle interconnection.
To improve overall health by modulation and control of the tissue-nerve cell innervation.

In this invention, a process is developed in which the PNS is accessed through the development of mechanical surface forces, including tensile and compressive forces, over the outer layer of the epidermis to induce electrochemical signals to the nervous system over a sustained period and graduated intensity for manipulation of the nervous system to improve functional capability of organs and other structures or responses. To improve the specificity, the treatment and therapies include the neuro-stimulation of the initiating source, such as activation of a transmitting nerve, as well as to the destination, such as an affected region in the gut in need of response, thereby forming a closed-loop control system of actuation to produce a measured result, as designed through examination of somatosensory maps to determine the optimal course of action: for example, the facial region has the highest density of receptors for the PNS, and therefore access to the nervous system can be most efficiently achieved through its neuro-stimulation. Moreover, the newly invented process, which induces a massively parallel neuromodulation of receptors to the PNS sustained over a significant time period, also influences the central nervous system through inherent pathways to improve attentive and meditation capability.

The field of use includes therapeutic applications, including implementations that are targeted at the treatment of internal processes. The duration of application is longer than previously employed, which is important for a sustained response. Thicker PVA films may be used, which can be pre-formed, e.g., by die coat methods. This yields lower cost, stronger, wider area film implementations. The films are applied, in addition to facial areas, to such locations as hands and joints, and the abdomen in an area proximate to the esophageal sphincter or cardia of the stomach. Films may be applied in multiple locations. Safety precautions, including UV sterilization of the films and use of therapeutic creams, are taken. Secondary stimuli, such as a cognitive or learning function, may accompany the treatment. The treatment may be repeated multiple times on a daily basis over an extended period. Measuring the response to the treatment determines such factors as how to alter the method, when to stop film applications, and ultimately the outcome or success.

### BRIEF DESCRIPTION OF THE DRAWINGS

FIG. 1 is a chart showing divisions of the nervous system and the locating point where this invention seeks access to control the response. The overall control sequence in accord with the present invention serves to influence the neurological system through stimulation of the sensory systems, essentially the mechanoreceptors, and then the influence of the stimulation on the peripheral nervous system, central nervous, and autonomic system, including the enteric system, with feedback to the sensory system provided through the autonomic system.

FIG. 2 graphically illustrates a chemical formula for the PVA polymer molecule used for the film formation.

FIG. 3 is a set of black-and-white (B/W) photographs of the PVA polymer films used for the applications in accord with the present invention.

FIG. 4 is a schematic side view of the placement of the PVA film on the epidural layer.

FIG. 5 is a schematic side view of the PVA film in place upon the epidural layer.

FIG. 6 is a schematic side view illustrating the detachment or washing away of the PVA film from the skin.

FIG. 7 is a schematic side view illustrating the application of the PVA film for the purpose of treatment of skin disorders, such as acne or open cuts.

FIG. 8 is a schematic side view illustrating the application of the PVA film for the purpose of delivery of topical medications, as well as tissue directed medications.

FIG. 9 is a schematic plan view illustrating treatment of the facial nerves by placement of the PVA film over facial areas.

FIG. 10 is a schematic view illustrating placement of PVA film for treatment of tissue targeted issues, such as gastric system, the motor system where blood flow is low, etc.

FIG. 11 is a flow sheet for an implementation of the method.

FIGS. 12A through 12C is a sequence of schematic side sectional views showing the procedure involving: (A) placement of film which comes into contact with surface; (B) dissolution of film and swelling coming into conformal contact with skin surface; and (C) evaporation of water solvent to achieve a thin solid film across the surface in conformal contact.

FIG. 13 is a chart of the distortion of the film when subjected to a water mist and then the solidification after an hour.

FIGS. 14A and 14B are B/W photographs that show (A) the application of the PVA film on top of the cream region after the completion of the drying period, wherein the transition from an initial state to a dried state took approximately forty five minutes and wherein the stretch/compressive forces were gradual; and (B) the standard case after the PVA film is removed, wherein it is noted that the skin area distorts to the interaction with the PVA film, as the nasolabial folds are noticeably reduced, as well as the flattening of the left and right cheek areas, a narrowing of the nasal region, and smoothing the area below the nose.

FIG. 15 is a schematic side sectional diagram of the sensory receptors that are triggered by PVA film application detecting pressure effects.

FIG. 16 is a schematic side sectional diagram showing how compression and shear stress impact the Ruffini endings within the dermis layer to trigger action potentials.

FIG. 17 is a B/W photograph of the film applied to one hand in comparison to normative situation indicating potential for improvement in straightening arthritic hands.

FIGS. 18A and 18B are computer display images of a single-point EEG measuring focus and relaxed state respectively (A) before and (B) with the application of the PVA film, noting an increase in both states of attention and meditation.

FIG. 19 is a B/W photograph of the film applied to a sore elbow, wherein stimulation of cutaneous afferents through the stress induced compressive and tensile forces from the drying and dried PVA film provides a topical analgesic effect.

### DETAILED DESCRIPTION

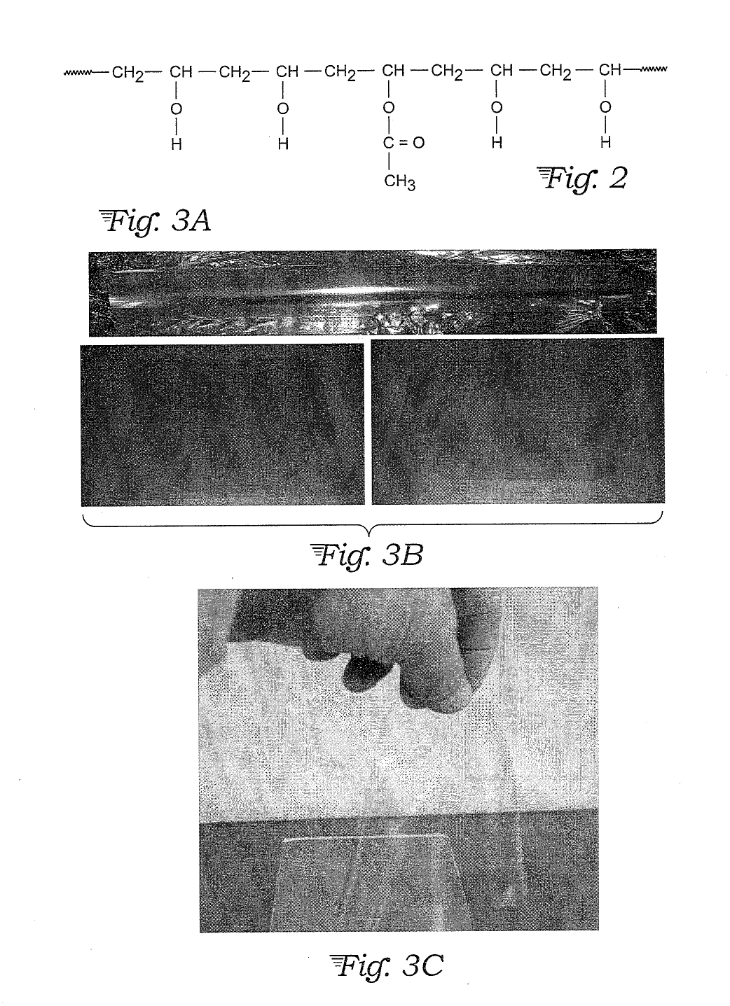

With reference to FIGS. 2 through 4, a water soluble film, which can be made of PVA, is presented to the surface of the skin, which can be pre-moistened with cream or other material of interest, followed by a water spray if desired. The PVA film consists of a blend of polyvinyl alcohol and water. The polyvinyl alcohol contains approximately 87% hydrolysis of acetate groups, a schematic of which is indicated in FIG. 2. To keep the costs low on the film forming material, the acetate groups remain unhydrolyzed in the final material. Within the film, glycerol can be added to improve elasticity as well as various anti-bacterial agents to promote stability.

The PVA films may be created either by a spin-coating method, or by a die-coating method (which is preferable for lower costs through higher throughput). For example, polyvinyl alcohol in a thickened format of 25% PVA in water may be cast onto glass plates, that may have nanostructures or microstructures to cast a pattern into the PVA thin film, or utilized directly. The material is processed to a 60 micrometer thin film, which is then cut into a desired size and shape, e.g. an oval shape at 6 inch×8 inch along its axis. The pH of the material in water is 5-7 and the viscosity as a 4% solution in water is 11-14 cP. Its ash property is less than 0.5% and is collected volatile condensable material is less than 5%. The molecular weight of the material is approximately 100,000 containing 5,000 units comprising the polymer chain. In FIG. 2, a photograph of the film and the various shapes for its intended use is documented. A photograph of the PVA film is shown in FIG. 3.

The approximately 60 micrometer thickness is selected to maximize displacement while also conforming, with a moderate level of water spray, to the surface layers of the skin, whether it be for example in the facial, neck, or torso regions. A thinner film, e.g. on the order of 20 to 40 microns thick, will rapidly form on the surface but ultimately provide less tension and compressive forces, while being more difficult to remove. Alternatively, thin films may be doubled to eliminate such issues.

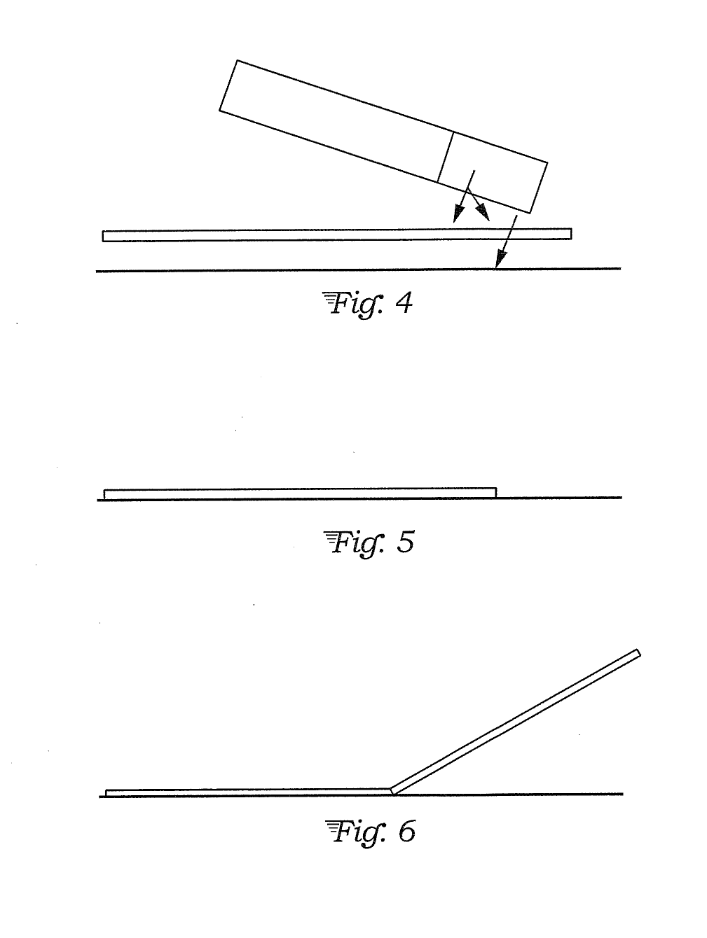

After the PVA film material has been placed upon target areas of the skin, as seen in FIG. 4, an applicator may then be used to lightly apply water to the external surface of the PVA film, thereby conformably contacting the skin, as indicated in FIG. 5, and removing any parts of the PVA film that are not in contact with the skin because, for example, the film extends beyond the area of the skin. The PVA film is then allowed to dry, which can take approximately 15-30 minutes, and then after a further 30-120 minutes the dried PVA film is physically removed from the skin, as shown in FIG. 6. It could also be removed by washing away with water. In addition, removal of the PVA film may be facilitated by first applying a buffering layer, such as cream, which is moistened with water and other nutrients, to the facial area or other area of interest.

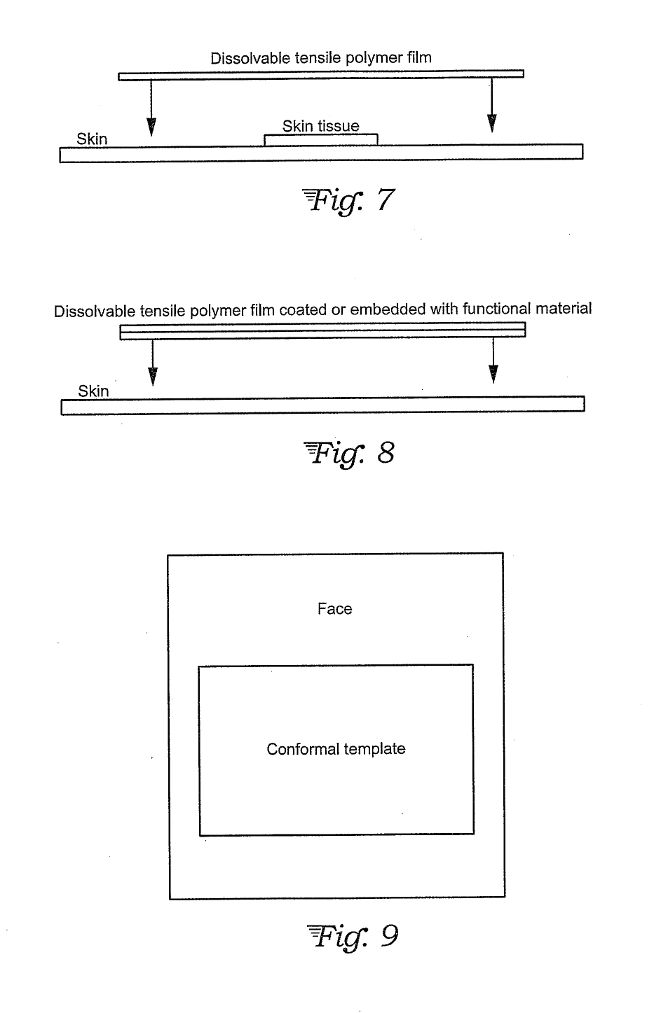

In FIG. 7, the use of the thin film for controlling certain skin disorders, such as acne, is shown. In this instance the thin film is brought into contact with the skin, and sealed with water. When the film undergoes a drying process, stresses are produced which induce stretching forces in the internal layers of the skin, and thereby sensory mechanoreceptors in the skin are activated, such that the necessary blood flow and nutrients, including white blood cells and other correcting chemicals, are sent to the affected region to correct the problem. The PVA film application has been applied to skin with acne, and found to adequately respond in terms of conforming to the surface, as well as its removal after application. In addition, the application of the PVA film to the tissues surrounding the acne seemed to help reduce the dimension of the affected regions.

Also in FIG. 7, in another application the thin film is used as a dressing for a cut. In this case, any bleeding that is present may locally dissolve a portion of the thin film. However, overall the film will apply compression to the wound area that can staunch the flow of blood and will stimulate mechanoreceptors in the region surrounding the cut that will stimulate a generally autonomic and reflexive response involving increased blood flow activity to the affected site around the cut, as well as pain reduction.

Tests of the PVA film application have been conducted on different skin types, including Caucasian, African-American, Latino, as well as male and female. No differences in response have been cited, and therefore it is not necessary to optimize the PVA formulation for different skin color. However, the amount of hair content on the skin does play a role in the application. If the hair is too thick, then the PVA film will not come into conformal contact with the skin to excite the mechanoreceptors, and hence not have its intended affect. Therefore, the hair on the targeted areas should be removed or avoided, and then the film applied for maximal effect.

As illustrated in FIG. 8, it is helpful in certain instances to associate a topical agent to the PVA film, and then bring the PVA film into contact with the surface, followed by water to achieve conformal contact. The topical agent can be one of several types, including skin moisturizers and anti-bacterial agents, as well as anti-acne formulas. This approach will permit a smooth and uniform appearance to the material, as well as the ability to entrap the material for high concentration, effective usage with sufficient solvent activity. It will also enable ease of removal.

In FIG. 9, placement of the PVA film over the surface contour of the face, as well as neck, will stimulate the facial nerves through its mechanoreceptors and thereby provide an immediate interaction with localized areas including the facial nerve itself in motor control.

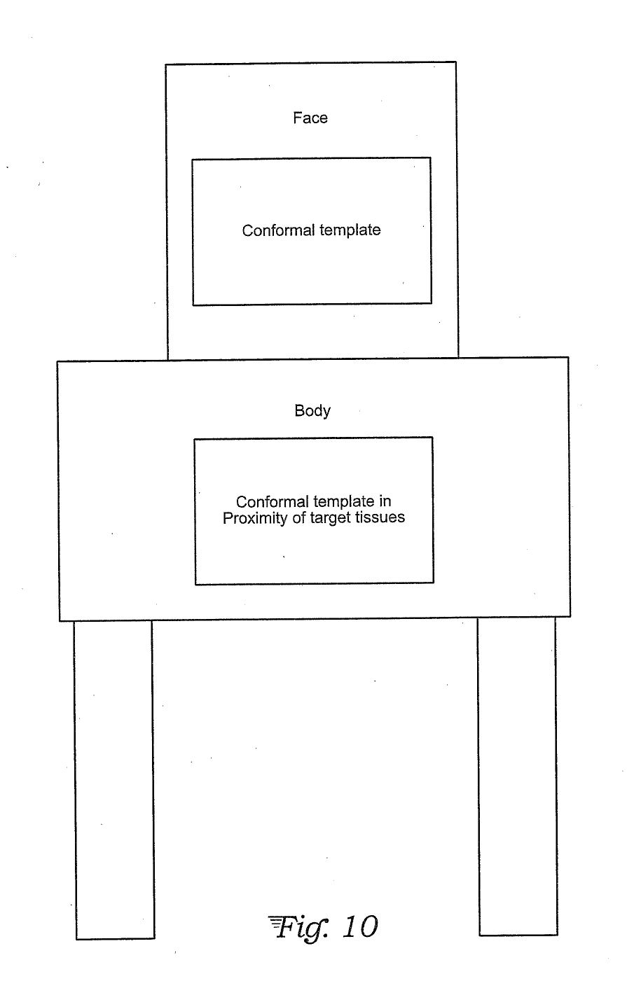

For control of the targeted tissue, the PVA film is placed on the face and then on the tissue region in which the control can be more fully recognized. In this scenario, the dense state of cutaneous afferents, essentially mechanoreceptors, of the facial region are modulated, and the targeted tissue location, such as the gut region, is also modulated, as represented in FIG. 10. The sensory system will send signals through the peripheral nervous into the autonomic nervous system, and promote the association of the sensory system of the gut to the coordinated activity of the CNS and PNS. This can address persistent problems such as GERD to achieve better health through improved innervation and its control.

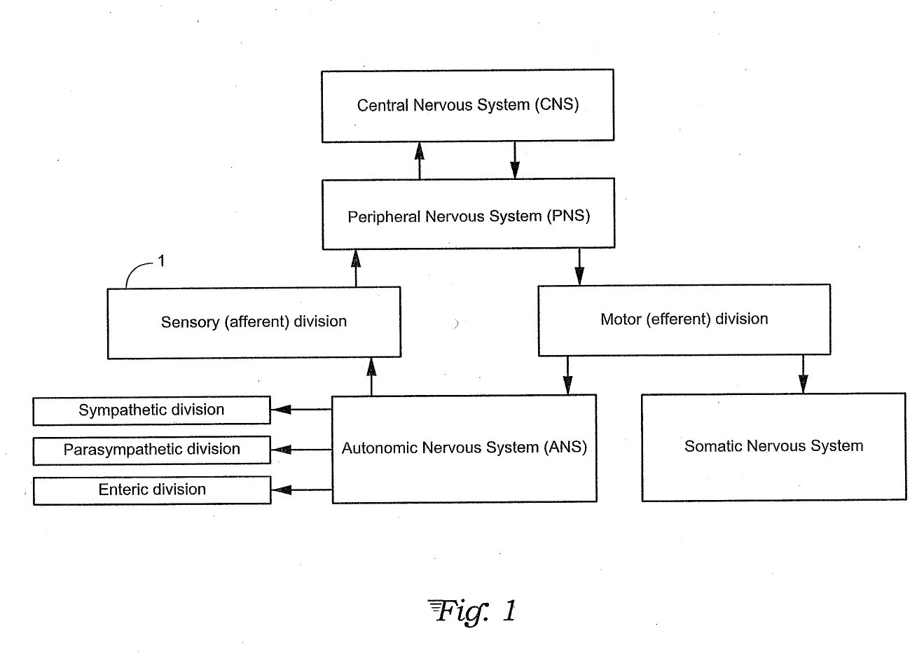

The theory for this approach is based on the interconnections of the nervous system, as indicated in FIG. 1. It shows that the sensory system is one of the only ways that one has access to control the inputs to the nervous system in order to achieve better and more logical control throughout the body. Moreover, the noninvasive nature of the approach permits the control procedure to be run on a daily basis, thereby improving internal adaptation through persistent learning.

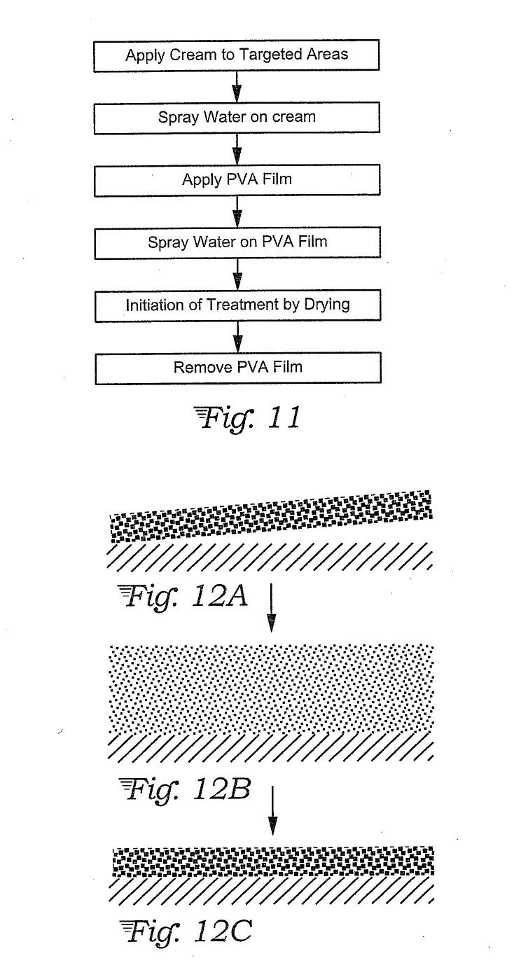

To modulate the mechanoreceptors of the peripheral nervous system, the PVA film is applied as follows: After a layer of cream is applied to the target surface followed by a misting water spray, the PVA film is placed on the target area, which will immediately undergo an adhesive effect as it comes into contact with the water on the surface of the cream. Further smoothing or time will enable the film to conform precisely to the contours of the surface. A second spray of water is applied, which can also be smoothed by touch to the skin surface. Afterwards, the water is allowed to evaporate, and after five minutes the surface tension will increase by the drying the PVA film. This sensation lasts over the next forty-five to sixty minutes while the film becomes nearly completely dried. Additionally, an hour or more can take place before the film becomes as dry as possible and reaches its limit of displacement. To continue the interaction, the muscles can be exercised to induce resistance force and further displacement of the skin layers. The mechanoreceptors pick up displacements as small as 10 nm. The process flow is indicated in FIG. 11.

To determine the extent of the drying PVA on skin, it is acceptable to touch the material to determine its degree of dryness, as there will be a liquid component remaining if the PVA film is not completely dried. Alternatively, if a water-absorbing material, such as paper, is applied to the surface and falls away from the film, then it is sufficiently dried. Moreover, there is a sensation that the film is tightening as it dries during the process, and when this process ceases, then the process is largely completed, although the stress field will maintain itself.

The film should complete its drying process before pain level is reached. During the removal process, which is done by peeling away the film, pain is possible if the cream layer thickness is not applied or is inadequate.

The wetting of the PVA film and subsequent shrinking of the PVA coating during the drying period, which is a film forming process, involves several steps in producing the time varying stress field across the targeted area. As indicated in FIG. 12A, the initial step involves the imperfect contact of the film onto the skin layer, followed by diffusion of water into the PVA film as well as the mixture of the cream layer, if present, with the PVA film and water. This action causes a swelling of the film layer, as indicated FIG. 12B, which shows that the applied water induces separation of the PVA polymer chains with a larger separation path. As indicated in FIG. 12C, evaporation of the water causes a coating of the PVA film on the skin, which is in conformal contact with the skin layers, thereby achieving an adhesive effect to the micro-structured surface that enables the stress forces to transmit through the skin layers to the receptor sites as displacement, resulting in firing of electrical action potentials.

The distances involved span to millimeters and are most noticeable at the edges of the film surface. If sufficient time is allowed the adhesion of the material onto the skin is insufficient to prevent the detachment of the film along the edge, which can become the initial point of removal.

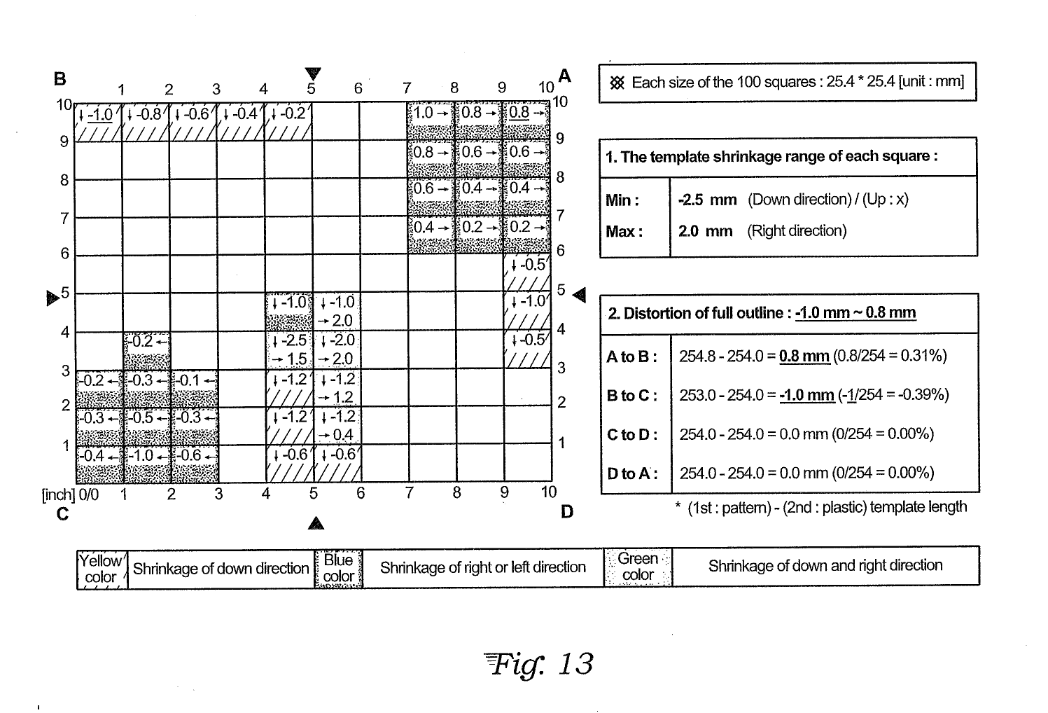

By placement of an adhering thin film soluble substrate, which contours to the surface topography, and undergoes phase transformation from a solid to gel back to solid, a dynamic stress field is established throughout the process, which induces time-varying displacement of the skin layers over a significant time period lasting at least two hours before converging to a stable surface overlay. In FIG. 13, the results of the free-standing film undergoing a dissolution and then solidification process is shown to distort an initial grid pattern with some areas shifted in a random direction dependent upon the spray process. It is expected therefore that the materials when applied to the skin layers will undergo various layers of thicknesses when applied to the water spray, however tension forces created by the process do follow general trends as further explained below.

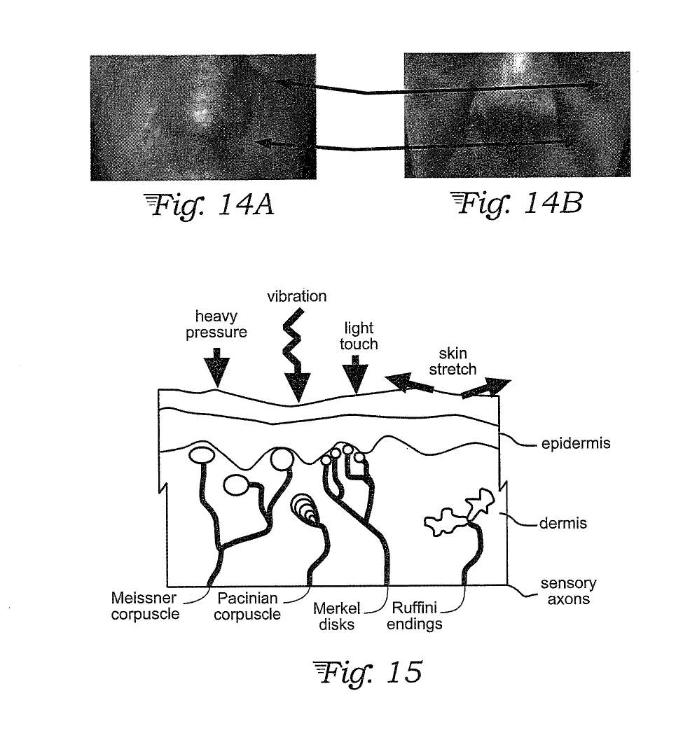

The response of the skin is dependent upon the location and geometry of the region being targeted, which is a function of the counter-resistance available to the applied force. For example, in the neck region, the effect is nominally one of stretching from the medial area outward, combined with a compressive force directed radially toward the central axis of the neck. For the face, it is nominally one of compressive smoothing, with a slight stretching outward to the edge. On the thigh and the stomach region, the film tends to induce a wave pattern, set-up by inward radiating forces. Photographs of these areas are indicated in FIGS. 14A and 14B. For the films tested from 20 microns to 60 microns thickness, damage of the skin was not observed over a two hour timeframe.

The effect of the PVA film dissolution and formation process induce forces including compressive stress, tensile stress, and shear stress propagating throughout the skin layers, impacting the mechanoreceptors, as shown in FIG. 15, to achieve a sensory response, registered through the CNS, PNS, and ANS. The tensile stress is a force that stretches and lengthens the mechanoreceptor and acts perpendicular to the stressed region. As the PVA film compresses the skin layers during the polymer-chain entanglement process experienced when drying, the sensory receptor is also compressed, undergoing a slight volume change to trigger a response. Deformation of the sensory receptor is achieved through shear stress, which tends to act in a single direction. It is noted that these forces are acting together and the mechanoreceptor is achieving a response as a result of their combined effects.

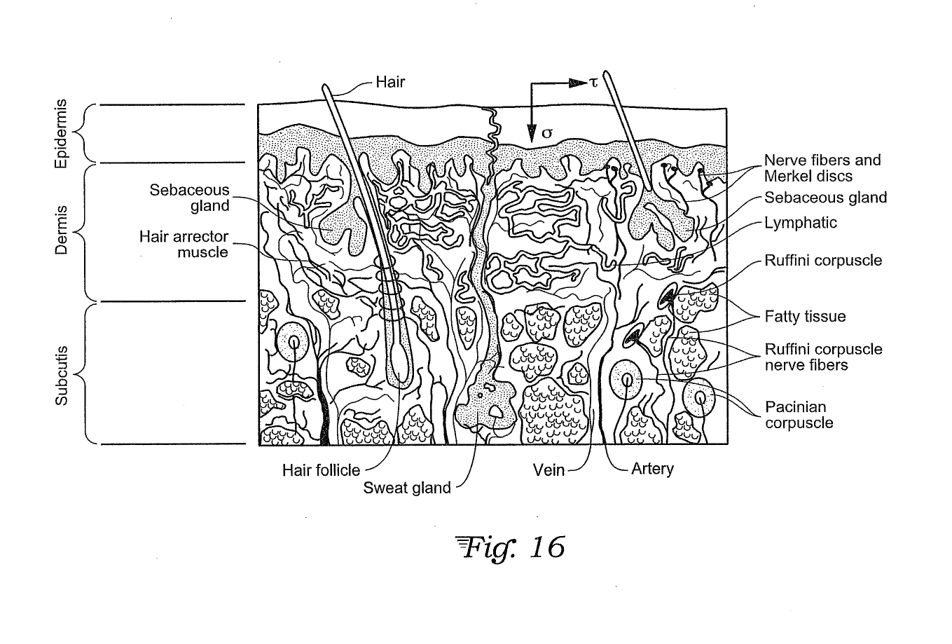

Nerve endings include mechanoreceptors detecting cell deformation such as stretching and bending, as well as touch, pressure and vibration; thermoreceptors detecting temperature; nociceptors detecting pain; photoreceptors detecting light; osmoreceptors detecting osmotic pressure of body fluid. As indicated in FIG. 16, it is primarily the mechanoreceptors that are affected by the application of the PVA film process, which include the displacement due to shear and tensile stress, as well as pressure deformation due to compressive stress. In addition, the motion of the skin due to muscle movement while the PVA film is administered, including its abnormal response due to the presence of the PVA film, will also trigger the mechanoreceptors.

In addition, the cellular deformation, and the interaction of the bodily fluids with the external environment, may induce vasodilation, which in turn will stimulate the somatosensory system at the localized site.

Advantages of the PVA application include the following: broad-area coverage, simultaneous activity, as well as slow persistent rate of in-plane stress field inducing a strain field of displacement, which collectively stimulate a large number of mechanoreceptors in a similar and sustained fashion, over a long period of time, inducing a range of low frequencies and amplitudes of electrical signals to the CNS, PNS, and ANS.

Other methods such as pinching will stimulate mainly high threshold receptors, nociceptors and phasic mechanoreceptors, and mechanical abrasion is rather limited in time and space, which will not provide for excitation of the tonic receptors.

TABLE 1

| | | Adaptation | Receptive | |
|---|---|---|---|---|
| Receptor | Sensation | rate | Field | Targeted |
| Free nerve | Itch, pain | Tonic, Phasic | Large, small | Yes |
| endings | | | | |
| Ruffini | Stretching, | Tonic (slow) | Large | Yes |
| endings | deep | | | |
| | pressure | | | |
| Merkel cells | Fine touch, | Tonic (slow) | Small | Yes |
| | pressure | | | |
| Meissner | Fine touch, | Phasic (fast) | Small | Yes |
| corpuscle | pressure | | | |
| Hair follicle | Rough touch | Phasic (fast) | Small | No |
| Krause bulbs | Vibration | Phasic | Small | No |
| | | (faster) | | |
| Pacinian | Vibration, | Phasic | Large | No |
| corpuscle | pressure | (fastest) | | |

In Table 1, the subset of the sensory system that is targeted for stimulation by the PVA film application is indicated. In particular, mechanoreceptors are targeted by the PVA film application. Due to the stretching and compression forces over an extended period of time, receptors that are slow to adapt will continue to produce action potentials during the entire process.

The procedure will last at least two hours, providing constant tension to the targeted sensory areas of the PNS. The rate at which the evaporation of the water during the drying process will determine the tension provided to the thin film, and hence the triggering of the somatosensory system will be dependent on the rate of evaporation. The time can be adjusted by forced convection of the evaporation process, and made faster drying, during which the firing of the sensory system will increase in frequency.

Based on a study in which the participant utilized the system on a daily basis for over a year, it is safe to apply on a day by day use, and recommended as such.

The treatment should be questioned for potentially halting depending upon the stimulation of the motor or sensory neurons of the facial nerve, which may result as a slight twitching in the muscle in the facial region along the line extending from the corner of the mouth to the ear. On one participant, this was noted after the first use of the method, but subsided and ultimately disappeared within two weeks. During that period, the treatment was executed every other day, and then returned to use on a daily basis.

### Applications

### (1) Support and Pain Relief

For supporting injured areas, the PVA film was tested on arthritic hands. The participant was a woman of 66 years age who suffered from arthritic hands, and an inability to keep the fingers straight. The materials were applied as follows: First cream was applied to both hands followed by a water spray, and then film approximately 60 microns thick and four inch square was applied to the upper portion of the hand; a second film was applied to the fingers and water spray was applied; followed by a third film applied to the upper hand, and then a final spray of water. After a period of drying, the participant expressed an improvement in finger straightening and a significant reduction in pain, and she continued to wear the film covering throughout the day.

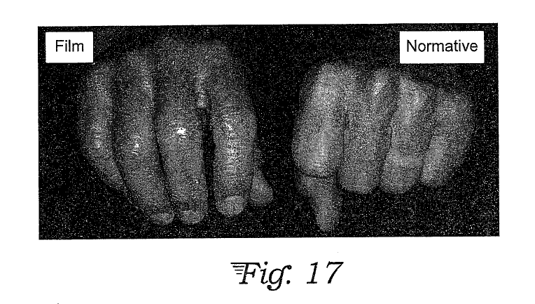

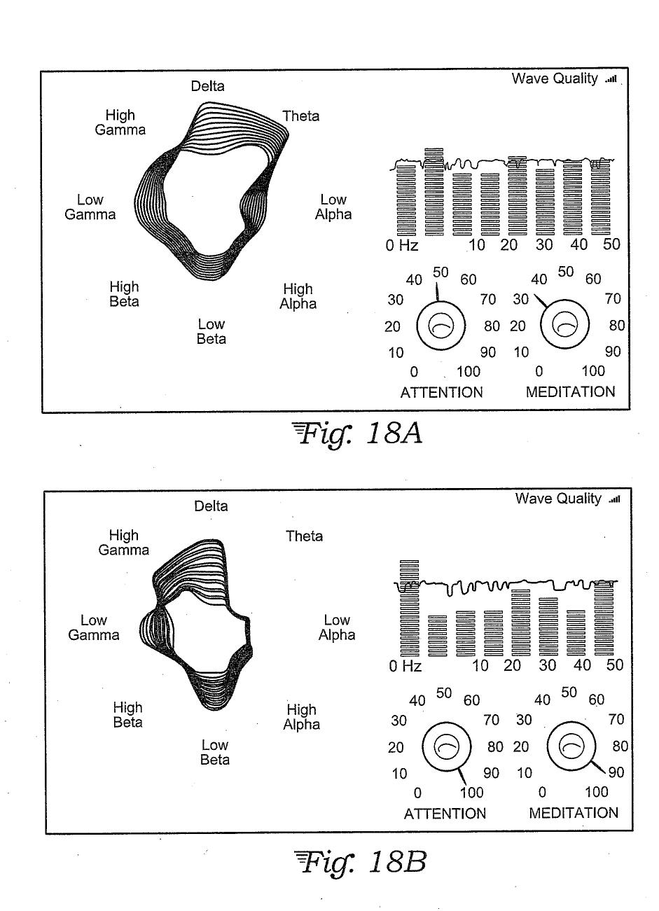

To evaluate the mechanism of improvement in arthritis, a similar procedure was followed on normative hands without arthritis, with one hand applied with the film, and the other without the film, producing the results of FIG. 17. The report is that the film provides support to the hands, deflecting the fingers upward in a straightening motion, as arthritic hands for the participant were deflected downward. The result is approximately 50% straightening from a clenched fist, which would require approximately 4″ (10 cm) of movement between open and closed, whereas with the film applied, there was 2″ (5 cm) of movement before the film tension become a significant force to overcome. With the film, it was possible to clench the hand fully without too much resistance, especially after breaking the adhesion about at the mid-joint of the hand.

In addition to arthritic conditions, the PVA film application is useful for common areas of injuries where elastic adhesive tape is nominally implemented, which would include carpal tunnel syndrome and wrist pain. The advantage of the technology would be ease of removal by simply peeling or washing with water, as well as continuous firming effect during the drying process.

Moreover, for pain relief, because the film provides a compressive and stretching mechanism, pain signals become dispersed and attenuated. This was tested on participants feeling pain in the elbow and in the gut area, with both situations reporting improvements. (It was also noted that the application of the film to the elbow had the added benefit of removing the roughness from the dried skin on the elbows; and therefore the PVA film application would be compatible with a cleansing routine for the elbow region, which tends to be rough as that region is generally without significant oil glands.)

### (2) Cognitive Skill Enhancement

The capability to improve cognitive skill by access of the PNS was evaluated by applying the film to the facial and neck regions, in order to achieve significant connection with the CNS. The methodology to apply the film consisted of applying a buffering layer of cream to permit easy removal, followed by water spray, and then multiple PVA films of approximately 60 μm thick across the entire facial and neck region, with the exception of the eyes, nasal openings and mouth. A final spray of water was applied, and after the material was smoothed across the region, a period of drying and film distortion of approximately sixty minutes was allowed, the results of a single point EEG are presented in FIGS. 18A and 18B (before and during the PVA film application, measured 45 minutes into the treatment period), indicating a significant increase in delta and beta brain waves while reducing theta brain waves. Interestingly in comparison to a normative state, the results suggest a heightened state of attention and meditation simultaneously. Consistent with studies on optimal cognitive states for learning as measured by EEG, this state achieved by the PVA film implementation is ideal for learning new functions at an accelerated rate when stimulated with a secondary learning source. Also interesting, in the absence of a secondary learning source, the participant in this case tended after fifteen minutes towards the onset of drowsiness or sleep.

### (3) GERD Therapy

In Table 2, the results of the application of the PVA film on a daily basis to the facial and neck regions in a method involving the implementation of cream, water, film and then water, in combination with the application of two four-inch (10 cm) square PVA films in direct contact without a buffering cream layer upon the gut region near the entrance point of the esophagus to the stomach to evaluate the capability of achieving a closed-loop control system to improve the condition of gastroesophageal reflux disease (GERD). The treatment was applied on a daily basis at approximately the end of the day lasting for about an hour each time. In particular, the table shows the reduction and elimination of famotidine, or any other antacid medication, following the application of PVA film therapy for GERD, which consisted of applying the film to the facial area, in addition to the gut area.

TABLE 2

| Date | Famotidine Fulfillment | Comment |
|---|---|---|
| Jan. 17, 2014 | 60 × 40 mg | Initially diagnosed |
| Nov. 3, 2014 | 60 × 40 mg | Twice daily start |
| Dec. 4, 2014 | 60 × 40 mg | Plus Antacids, Pepcid |
| Jan. 12, 2015 | 60 × 40 mg | Treatment started |
| | | February 2015 on daily basis |
| Apr. 6, 2015 | 60 × 40 mg | 42 tablets remaining June 2016 |

The participant was diagnosed in early 2014 and prescribed famotidine, which reduces the amount of acid in the stomach, but does not resolve the basic problem and only is a way to manage the effects of GERD. A prescription was assigned of 60 units of 40 mg each, which were to be taken twice daily. During the initial phase, the famotidine was taken sparingly with pain managed through an antacid liquid (Al hydroxide, Mg hydroxide, Simethicone). Around the fall of 2014, the pain become significant enough were the participant began taking famotidine twice a day as prescribed, including continuing with the antacid as well as OTC medications, such as Pepcid. Around February 2015, the use of the PVA film system was implemented on a daily basis, and a dramatic reduction in famotidine occurred as noted in Table 2, resulting in the elimination of any requirement for an antacid of any type. The PVA film system treatment was implemented for a year and the participant reports no symptoms of GERD throughout this one year period, and takes no medications of any sort. We believe that the film system enabled improved coordination of the nervous system to control the opening of the connection point of the esophageal area and stomach to prevent backfill of stomach acid, as well as improved muscle tone in this region.

### (4) Vasodilator (Inflammation, Acne, Post-Surgical Treatment, Injuries)

For controlling inflammation, which would be the case for post-surgical procedures or for skin conditions such as acne, the film treatment system was evaluated for its ability to function as a vasodilator, increasing the blood flow to the affected site. This was evaluated for two acne related cases and found to improve the overall condition, and was evaluated for one post-surgical treatment in which the inflammation was found to increase with the application of the film system.

### (5) Appetite Suppression

When applying the film system, it is apparent that the appetite becomes suppressed because of the distraction of the stimulation provided by the film system resulting in a cessation of hunger pains, as well as the inability to conveniently eat. For a normative 60 mm opening of the mouth without the film, it is possible to conveniently open the mouth only 15 mm, which significantly reduces the desire to eat. Consequently, the film system device and implementation process is useful in a weight loss regiment. This was tested with a participant who applied the procedure at the end of the day for approximately one hour, which abated the tendency towards eating at that period, resulting in weight reduction and improved health.

### (6) Relief of Joint Soreness

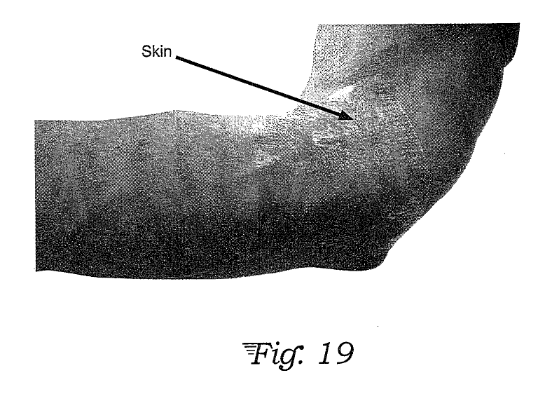

In FIG. 19, the application of the PVA film to the inner elbow area is demonstrated in response to soreness as felt in the joint area. By neuro-stimulation of the nervous system of the area proximate to the area associated with soreness, and through vasodilation of that area as noted by a change in dermal color, the discomfort and pain is reported to be attenuated. The film was left in place for approximately two hours, of which the first thirty to forty-five minutes, the film transitioned from a gel state to a solid state. The subsequent period included sustained tightening of the film along the surface of the skin, resulting in a stimulation of the area, and a corresponding reduction in discomfort. The film was subsequently removed by simply peeling.

What is claimed is:

1. A process for targeted stimulation of the nervous system, comprising:
applying a water-soluble polymer over at least one target area of skin in a manner so as to cause partial adhesion of a gel-state polymer film to micro-scale skin folds in at least one target area;
allowing a period of drying of the polymer film to a solid state with a corresponding dimensional change of the film that induces stresses throughout surface areas of the skin in proximity to the polymer film sufficient to stimulate sensory receptors of at least the peripheral nervous system; and
leaving the dried polymer film on the skin for a specified sustained duration.

2. The process as in claim 1, wherein the water-soluble polymer is applied in a thick liquid format and then dried.

3. The process as in claim 1, wherein the water-soluble polymer is applied as a preformed film sheet over wet skin.

4. The process as in claim 3, wherein the target area of the skin is sprayed with water prior to applying the film.

5. The process as in claim 3, wherein a thin layer of cream is first applied to the target area prior to applying the film.

6. The process as in claim 5, wherein the cream has a composition with sufficient water to wet the applied film.

7. The process as in claim 5, wherein, after applying the cream, the target area is sprayed with water prior to applying the film.

8. The process as in claim 1, wherein the applied water-soluble polymer is sprayed with water sufficient to cause a transition from a solid state to a gel state.

9. The process as in claim 8, wherein the polymer material is smoothed by hand when in the gel state.

10. The process as in claim 1, further comprising applying a second water-soluble polymer film at least overlapping the gel-state polymer film adhering to the target area of the skin.

11. The process as in claim 1, further comprising removing the polymer film after a specified period of time on the target area.

12. The process as in claim 11, wherein the film is removed by peeling.

13. The process as in claim 12, wherein peeling of the film from elbow or knee target areas removes scaled skin thereby causing cleansing.

14. The process as in claim 11, wherein the film is removed by dissolution with water.

15. The process as in claim 11, wherein the water-soluble polymer comprises polyvinyl alcohol.

16. The process as in claim 1, wherein the at least one target area comprises any one or more of facial areas, neck, hands, feet, torso, or abdomen.

17. The process as in claim 1, wherein the water-soluble polymer is applied to multiple target areas.

18. The process as in claim 1, wherein the induced stresses comprise area-wise compression upon the skin surface in the target area.

19. The process as in claim 1, wherein the induced stresses include local stretching of the skin surface.

20. The process as in claim 1, wherein the sensory receptors stimulated by the induced stresses in the target area are mechanoreceptors.

21. The process as in claim 1, wherein the sensory receptors stimulated by the induced stresses in the target area are nociceptors.

22. The process as in claim 1, wherein stimulation of sensory receptors in at least one target area of skin enables a noninvasive neuromodulation of the peripheral nervous system with a specified therapeutic effect.

23. The process as in claim 22, wherein stimulation of mechanoreceptors in one or more target hand areas reduces pain associated with arthritis.

24. The process as in claim 22, wherein stimulation of mechanoreceptors over a broad area proximate to a joint area reduces soreness.

25. The process as in claim 22, wherein broad area stimulation of cutaneous afferents in one or more target areas controls pain.

26. The process as in claim 22, wherein broad area stimulation of mechanoreceptors in one or more target facial areas induces a relaxed state sufficient to achieve sleep or meditation.

27. The process as in claim 22, wherein broad area stimulation of mechanoreceptors in one or more target facial areas induces an attentive state sufficient to improve cognitive function.

28. The process as in claim 22, wherein stimulation of mechanoreceptors associated with the nervous system in any one or more of facial regions, neck regions or in abdominal regions of the skin proximate to the esophageal sphincter or cardia of the stomach contribute to a reduction in gastric reflux.

29. The process as in claim 22, wherein stimulation by compressive forces of mechanoreceptors of full facial and neck areas suppresses appetite.

30. The process as in claim 1, wherein the induced stresses upon surface areas of the skin in proximity to the polymer film selectively induce inflammation control in compressed areas and vasodilation and relief of hydrostatic pressure in cellular structures in stretched areas so as to promote healing in post-surgical treatment.
